(19) United States
(12) Patent Application Publication
(10) Pub. No.: US 2025/0114381 A1
(43) Pub. Date: Apr. 10, 2025

(54) Cannabinoid Composition and Uses Thereof

(71) Applicant: Brains Bioecutical Corp, Vancouver (CA)

(72) Inventors: Naresh R. Thumati, Vancouver (CA); Jason Hastings, Carson City, NV (US); Arthur Jaffee, Broomfield, CO (US); Ricky Brar, Vancouver (CA); Kevin Coft, Vancouver (CA)

(73) Assignee: Brains Bioceutical Corp, Vancouver (CA)

(21) Appl. No.: 18/376,679

(22) Filed: Oct. 4, 2023

(51) Int. Cl. A61K 31/00; A61K 31/01; A61K 31/015

(52) U.S. Cl. CPC A61K 31/658; A61K 31/01; A61K 31/015

(57) ABSTRACT

Cannabinoids are increasingly found to have beneficial effects on the human body, even without the intoxicating effects from THC. Herein we describe a cannabinoid composition of mixed cannabinoids and terpenes. The described cannabinoid composition can be used for improvement of sleep quality, for pain management, and for stimulation of neurogenesis. The typical cannabinoid composition is 90.0% by mass cannabidiol, 4.0% by mass cannabigerol, 2.0% by mass cannabichromene, 2.0% by mass cannabinol, 1.5% by mass beta caryophyllene, and 0.5% by mass myrcene.

### BACKGROUND

### Field of the Invention

The present invention relates generally to compositions of cannabinoids and terpenes. More specifically, the present invention relates to compositions of cannabinoids, such as cannabidiol, cannabinol, cannabichromene, and cannabigerol combined with terpenes, such as beta caryophyllene and myrcene that can be used for nutraceutical applications.

### Description of the Related Art

Cannabinoids are beginning to show many nutraceutical benefits. Studies are showing cannabinoids are having positive effects on quality of sleep, pain management, neurogenesis, and a number of other factors. Quality sleep and pain management are requirements of a healthy lifestyle; neurogenesis in adults can improve memory, improve learning, and may play a role in stress response and emotion.

Good quality sleep is a critical component of a healthy lifestyle. Studies have shown that getting less sleep than normal tends to leave individuals tired, irritable, less creative, less emotionally receptive, and less open to new ideas. Subjects suffering from insomnia, diagnosed or otherwise, are at risk for poor physical and mental performance during waking hours. In combination with an unhealthy lifestyle, poor sleep can result in declines in the workplace, declines in the familial space, and general malaise that is difficult to pinpoint and treat.

A 2009 study of sleep disturbance in obese populations reported that 84.8% of obese patients reported at least one sleep disturbance symptom. In addition, men and women were shown to exhibit different symptoms. Women were found to be affected most by daytime sleepiness, night sweats, difficulties falling asleep, and difficulties staying asleep. Men were affected most by fatigue, night sweats, and difficulty falling asleep.

A variety of benefits exist from getting proper amounts and quality of sleep. Chronic sleep debt may lead to the development of mood disorders like depression and anxiety. Lack of sleep can lead to type II diabetes, primarily related to the handling of blood glucose. Long standing sleep deprivation was also associated with increased heart rate, blood pressure, and inflammation markers, leading to increased incidence of heart disease. In general, sleep plays a crucial role in healthy regulation of biological processes, and disturbance of that regulation may result in declines in health.

The most common suggestions related to improvement in sleep are pharmaceuticals, improving sleep hygiene, improving diet, and increasing exercise. While improving sleep with the use of pharmaceuticals is the most directly effective, it is not without risks.

Of the many pharmaceuticals are taken to assist with sleep, the most frequently taken are hypnotics. Hypnotics run a number of risks, and even negative side effects related to sleep. While hypnotics improve the ability to fall asleep quickly, a number interfere with REM sleep. Many hypnotics have high risk of overdose or substance dependence with prolonged use, such as barbiturates and benzodiazepines. Other pharmaceuticals taken to assist with sleep are used off-label, and have limited clinical effectiveness, such as melatonin and antihistamines.

Sleep hygiene is a behavioral and environmental practice to assist with quality of sleep. The primary recommendations are to establish a sleep schedule, reducing naps, limiting activities prior to bed, reducing consumption of alcohol and stimulants in the hours prior to bedtime, and having a peaceful, comfortable, and dark sleep environment. While this practice has been commonplace since the 1970s, recent studies have shown that the effectiveness of sleep hygiene is limited for the general population.

Studies are showing that diet and nutrition are affecting sleep quality. Studies have shown that a diet high in carbohydrates promoted reduced time of wakefulness in bed prior to sleep as compared to a diet high in fat, mixed micronutrients and macronutrients are needed to promote quality sleep, varied diet of fresh fruits and vegetables, whole grains, and low in saturated fat may be optimal for improving sleep quality. However, there are no existing clinical studies on long-term dietary practices as they relate to sleep quality.

Diet regimes, require substantial commitment to a restrictive diet, and to maintain the commitment for an extended period of time. The early stages of diets can be particularly challenging for those undertaking them as the restrictive nature of the diet requires additional time commitments for determining which, and how much, foods should be consumed. Additionally, the “on diet” meals may be not to the palette of those undertaking the diet as the flavor profile is likely very different from the flavor profile of the typical meal consumed when not on the diet. While dieting, social events also present frequent, and occasionally awkward, challenges as it is frequent that dieting at a social event will be difficult at best. This results in convenient opportunities for “cheat days” which undermine the results of the diet.

How exercise and physical activity improves sleep is currently unknown in medical science. However, it is known that moderate aerobic exercise increased the amount of deep sleep achieved. Exercising too late, though, can have a negative impact on sleep. Aerobic exercise releases endorphins which keep you awake. Additionally, exercising raises core body temperature, which also signals to the body that it is time to be awake.

Furthermore, exercise requires substantial commitment in both financial and temporal terms. These are frequent barriers. Financial investments range from one-time costs such as home equipment to recurring costs such as gym memberships and things such as new athletic shoes. The temporal investment required to maintain the commitment to exercise alone is substantial and well-studied outside of this patent application. The personal discipline required to maintain the financial and temporal commitment to an exercise regimen, in the face of muscular soreness and overall fatigue, is another barrier to individuals wishing to improve sleep quality through exercise.

Pain management is typically achieved via pharmaceuticals, physical manipulation of the body, or natural remedies. Managing pain with pharmaceuticals is the most effective, it has a number of risks.

Pharmaceutical pain management comes in a number of forms, from over-the-counter products such as ibuprofen and aspirin to prescription narcotics. While the over-the-counter products are generally safe, they are less effective than prescription narcotics, and also come with their own risks. Most over-the-counter pain management products have liver toxicity issues if taken in too large of dose or for too long of a time period. Aspirin functions as a mild blood thinner, which can lead to other health issues. Many drugs have known interactions with over-the-counter pain management products, some of which are quite severe.

The risks of prescription narcotics are well known and well documented, ranging from substance abuse and addiction to overdose and death.

Physical manipulation of the body can help manage pain for some sources of pain. Massage and chiropractic care, for example, are only particularly useful for back and neck pain. When performed incorrectly both have risks of more severe pain and other serious injury in addition to not functioning to manage the pain for which the treatment was sought. Exercise can be used for management of pain, yet also can lead to more pain. The downsides of exercise for some individuals are outlined above.

Natural remedies, including herbal remedies, meditation, and mind-body therapies, have some evidence of pain management, but none are well studied. All evidence currently for natural remedies is anecdotal. More studies are needed to establish the true value of natural remedies for pain management.

Neurogenesis is the biological process of the creation of new neurons within the brain. The vast majority of neurogenesis in humans occurs in utero and the first two years of life. Stimulation of neurogenesis in adults is thought to have benefits in memory, learning, stress response, and emotion. Microdoses of psilocybin have shown to stimulate neurogenesis in laboratory animals. Some cannabinoids have some evidence of stimulation of neurogenesis in the hippocampus, but other cannabinoids have had no effect on neurogenesis.

### SUMMARY

In an illustrative embodiment of the invention, the cannabinoid composition is 90.0% by mass cannabidiol (CBD), 4.0% by mass cannabigerol (CBG), 2.0% by mass cannabichromene (CBC), 2.0% by mass cannabinol (CBN), 1.5% by mass beta caryophyllene, and 0.5% by mass myrcene.

### DETAILED DESCRIPTION OF EMBODIMENTS

In the following description, for purposes of explanation and not limitation, details and descriptions are set forth in order to provide a thorough understanding of the present invention. However, it will be apparent to those skilled in the art that the present invention may be practiced in other embodiments that depart from these details and descriptions without departing from the spirit and scope of the invention.

In an illustrative embodiment of the invention, the composition is at least one cannabinoid with at least one terpene. The at least one cannabinoid could be cannabidiol, cannabinol, cannabichromene, or cannabigerol, or some combination thereof. The at least one terpene could be beta caryophyllene, myrcene, or both. In the embodiment where all four of the named cannabinoids are present, the cannabidiol could be between 50.0% and 95.0% by mass, the cannabinol could be between 0.1% and 5.0% by mass, the cannabichromene could be between 0.1% and 5.0% by mass, and the cannabigerol could be between 1.0% and 8.0% by mass. In the embodiment where both terpenes are present, the beta caryophyllene could be between 0.1% and 3.0% of the composition by mass and the myrcene could be between 0.1% and 1.5% of the composition by mass.

In another embodiment of the invention, the composition is 90.0% cannabidiol by mass, 4.0% cannabigerol by mass, 2.0% cannabichromene by mass, 2.0% cannabinol by mass, 1.5% beta caryophyllene by mass, and 0.5% myrcene by mass.

Any of these embodiments can be used for improvement in quality of sleep, as a pain management solution, or for the promotion of neurogenesis.

What is claimed is:

1. A composition comprising:
at least one cannabinoid; and
at least one terpene.

2. The composition of claim 1, wherein the at least one cannabinoid is selected from the group consisting of cannabidiol, cannabinol, cannabichromene, and cannabigerol.

3. The composition of claim 1, wherein the at least one terpene is selected from the group consisting of beta caryophyllene and myrcene.

4. The composition of claim 1, wherein the at least one cannabinoid is cannabidiol, cannabinol, cannabichromene, and cannabigerol.

5. The composition of claim 4, wherein the cannabidiol is between 50.0% and 95.0% of the composition by mass, the cannabinol is between 0.1% and 5.0% of the composition by mass, the cannabichromene is between 0.1% and 5.0% of the composition by mass, and the cannabigerol is between 1.0% and 8.0% of the composition by mass.

6. The composition of claim 1, wherein the at least one terpene is beta caryophyllene and myrcene.

7. The composition of claim 6, wherein the at beta caryophyllene is between 0.1% and 3.0% of the composition by mass and the myrcene is between 0.1% and 1.5% of the composition by mass.

8. The composition of claim 1, wherein the composition is used as a sleep aid.

9. The composition of claim 1, wherein the composition is used as a pain management solution.

10. The composition of claim 1, wherein the composition is used to promote neurogenesis.

11. A composition comprising;
cannabidiol;
cannabinol;
cannabichromene; and
cannabigerol.

12. The composition of claim 11, wherein the cannabidiol is between 50.0% and 95.0% of the composition by mass, the cannabinol is between 0.1% and 5.0% of the composition by mass, the cannabichromene is between 0.1% and 5.0% of the composition by mass, and the cannabigerol is between 1.0% and 8.0% of the composition by mass.

13. The composition of claim 11, further comprising at least one terpene.

14. The composition of claim 14, wherein the at least one terpene is beta caryophyllene and myrcene.

15. The composition of claim 14, wherein the beta caryophyllene is between 0.1% and 3.0% of the composition by mass and the myrcene is between 0.1% and 1.5% of the composition by mass.

16. The composition of claim 11, wherein the composition is used as a sleep aid.

17. The composition of claim 1,1 wherein the composition is used as a pain management solution.

18. The composition of claim 11, wherein the composition is used to promote neurogenesis.

19. A composition comprising;
cannabidiol, where the composition is 90.0% cannabidiol by mass;
cannabigerol, where the composition is 4.0% cannabigerol by mass;
cannabichromene, where the composition is 2.0% cannabichromene by mass;
cannabinol, where the composition is 2.0% cannabinol by mass;
beta caryophyllene, where the composition is 1.5% beta caryophyllene by mass; and
myrcene, where the composition is 0.5% myrcene by mass.

20. The composition of claim 19, wherein the composition is used as a sleep aid.

21. The composition of claim 19, wherein the composition is used as a pain management solution.

22. The composition of claim 19, wherein the composition is used to promote neurogenesis.
